(19) United States
(12) Patent Application Publication
(10) Pub. No.: US 2019/0282734 A1
(43) Pub. Date: Sep. 19, 2019

(54) PRODUCT AND PROCESS FOR THE PREPARATION OF AN INJECTABLE COMPOSITION BASED ON CHITOSAN AND BIOGLASSES, INTENDED FOR THE REGENERATION OF BONE TISSUE

(71) Applicants: UNIVERSITE CLAUDE BERNARD LYON I, VILLEURBANNE (FR); INSTITUT NATIONAL DES SCIENCES APPLIQUEES DE LYON, VILLEURBANNE CEDEX (FR); UNIVERSITE JEAN MONNET, SAINT ETIENNE, SAINT ETIENNE (FR); CENTRE NATIONAL DE LA RECHERCHE SCIENTIFIQUE, Paris (FR); UNIVERSITE DE MONTPELLIER, MONTPELLIER (FR)

(72) Inventors: Alexandra MONTEMBAULT, SAINT ETIENNE (FR); Laurent DAVID, LYON (FR); Thierry DELAIR, LYON (FR); Solene TADIER, LYON (FR); Laurent GREMILLARD, SAINT ETIENNE (FR); Annelise FAIVRE, PRADES LE LEZ (FR); Florence DESPETIS, PRADES LE LEZ (FR)

(21) Appl. No.: 16/301,908

(22) PCT Filed: May 19, 2017
(86) PCT No.: PCT/FR2017/051231

§ 371 Date: Nov. 15, 2018

(30) Foreign Application Priority Data
May 19, 2016 (FR) 16 54470

(51) Int. Cl. A61L 27/46; A61L 27/20; A61L 27/10

(52) U.S. Cl. CPC A61L 27/46; A61L 27/20; A61L 27/52; A61L 2430/02; A61L 2400/06; A61L 27/10

(57) ABSTRACT

The invention relates to a product intended for the preparation of an injectable composition containing, separated from one another, at least one first and one second composition(s), and in which: —the first composition, referred to as A, is an aqueous solution of chitosan with a pH in the range extending from 3 to 7, and —the second composition, referred to as B1, corresponds to particles of one or more bioactive glass(es), said product not containing glycerophosphate; and also a process for preparing an injectable composition, the corresponding injectable compositions and devices for packaging a product according to the invention.

The present invention aims to propose a two-component product dedicated to the preparation of an injectable formulation, the application of which concerns the regeneration of bone tissue after injection into the body of a subject. More precisely, the envisaged fields of application are the treatment of bone loss in orthopaedics or dentistry.

Bioactive glasses, also called bioglasses, are known as ceramic or glass-type materials capable of developing on their surface, in the presence of an aqueous medium, or at their interface with a body tissue, a layer of crystallized carbonated calcium phosphate hydroxyapatite, thus producing a biologically useful response. This hydroxyapatite layer is, by its composition, similar to the mineral phase of bone.

The concept of bioactive glasses includes both glasses in the strict sense of the term and partially crystallised glass-ceramic materials, i.e. containing both at least one amorphous phase formed of bioactive glass and at least one crystalline phase, the amorphous phase not necessarily being the majority. For more details on bioactive glasses, refer to the book “BIOMATERIALS SCIENCE, An Introduction to Materials in Medicine, 2d Edition by Buddy D. Ratner, Allan S. Hoffman, Frederick J. Schoen, Jack E. Lemons, Elsevier Academic Press, chapter 2.10 Ceramics, Glasses, and Glass-Ceramics 153 Larry L. HENCH and Serena BEST” (2004), as well as to the book “Bioactive glasses, Materials, properties and applications, Edited by Heimo O. Ylänen, Woodhead Publishing” (2011).

Bioactive glasses can be prepared in different ways, notably as described in Les Techniques de I'Ingénieur (n4955 “Verres bioactifs”, published 10 Apr. 2014). Bioactivity can be tested according to ISO 23317.

In the prior art, attempts to combine bioactive glasses with chitosan, a polysaccharide known to be biocompatible, bioresorbable, bacteriostatic and fungistatic, have been proposed. Oudadesse et al. (Jebahi S, Saoudi M, Farhat L, Oudadesse H, Rebai T, Kabir A, El Feki A, Keskes H. Effect of novel curcumin-encapsulated chitosan-bioglass drug on bone and skin repair after gamma radiation: experimental study on a Wistar rat model, Cell Biochem Funct, 2015, 33, 150-159) have worked on the elaboration of bioglasses/chitosan composite materials for applications in bone reconstruction and for wound healing. The authors propose to mix a chitosan solution prepared in acidic medium and a bioglass suspension in acidic medium, and then to increase the pH of the resulting mixture by adding soda. The addition of soda induces gelation of the mixture, which is then washed and freeze-dried.

Other solutions of the prior art propose to use a chitosan/bioglass mixture containing glycerophosphate, which, at 37° C., forms a gel with the chitosan solution containing the bioglass. Particular mention may be made of D. S. Couto, Z. Hong, J. F. Mano Development of bioactive and biodegradable chitosan-based injectable systems containing bioactive glass nanoparticles. Acta Biomaterialia 5 (2009) 115-123.

These different techniques are unsatisfactory because they either do not lead to injectable solutions or they use cross-linking agents that pose toxicity problems.

In this context, the present invention aims to propose a solution to these problems and concerns a product intended for the preparation of an injectable composition. The product according to the invention must allow simple and rapid preparation of an injectable composition suited to the regeneration of bone tissues.

The invention concerns a product, intended for the preparation of an injectable composition, containing, separately from each other, at least one first and one second composition, and wherein:
the first composition, called A, is a chitosan aqueous solution with a pH in the range of 3 to 7, and
the second composition, called B1, corresponds to particles of one or more bioactive glass(es),

said product not containing glycerophosphate.

The product according to the invention may also contain a composition C corresponding to water or to an aqueous solution (in particular, of buffer solution or culture medium type) with a pH in the range of 5.5 to 8, preferably in the range of 6.8 to 7.2. In this case, composition C will preferably be intended to be mixed with composition B1 to form an intermediate composition B2 corresponding to an aqueous suspension of particles of at least one bioactive glass, the pH of this suspension being in the range of 5.5 to 8.

Advantageously, the product according to the invention consists exclusively of compositions A and B1, or even compositions A, B1 and C.

Preferably, the product according to the invention does not contain genipin, glutaraldehyde or formaldehyde. More generally, the product according to the invention does not contain cross-linking agent.

When it is said that the product according to the invention does not contain a component, it means that said component is not present in composition A, or in composition B1, or in any other composition C that the product may contain.

In the context of the invention, the mixing of compositions A and B1, or of compositions A and B2, or even of compositions A, B1 and C, gives an injectable composition, which will then lead to the formation of a chitosan physical gel. No cross-linking agent leading to a chemical gel is used. The solution according to the invention is therefore less toxic than previous solutions using such cross-linking agents, since no cross-linking agent that would then remain in the body is present.

In the context of the invention, the approach is totally different from previous solutions using glycerophosphate, since it is the bioactive glass particles that are used to obtain, in the short term, gelation of the chitosan. The product according to the invention leads, after compositions A and B1, or compositions A and B2, or even compositions A, B1 and C, are mixed, to an injectable composition which can be used for the regeneration of bone tissue, and therefore, in orthopaedics and dentistry. Such an injectable composition corresponds to a suspension of particles of at least one bioactive glass in a chitosan aqueous solution. The mixing of compositions A and B1, or of compositions A and B2, or even of compositions A, B1 and C, leads, first, to an injectable composition, which will evolve over time to lead to a gelation of the chitosan, due to cation exchange between the particles of bioactive glass(es) and the chitosan. In the context of the invention, it is thus possible to obtain a gelation that occurs in situ, and therefore in vivo, after injection of the composition into the body, which allows the bioactive glass particles to remain at the implantation site.

The resulting injectable composition of bioactive glass(es) has an improved tolerance compared with a simple suspension of bioactive glass(es), due to the presence of chitosan. Indeed, conventional bioactive glasses, when used alone, cause a significant increase in pH at the implantation site, and are associated with a certain toxicity, or even tissue necrosis. The presence of the chitosan aqueous solution, at the selected pH, will allow the resulting increase in pH to be controlled, in particular, within the body. Similarly, the chitosan solution, used alone, would have a certain toxicity to the body due to its acidic nature. The combined use of the two makes it possible to reach pH values in the body in the range of 6.5 to 7.5, notably from 6.8 to 7.4, as function of the initial pH of the chitosan solution in particular, which is well suited to injectable implants.

The initial state (i.e. shortly after compositions A and B1, or compositions A and B2, or even compositions A, B1 and C, are mixed) of the injectable composition, combining chitosan and bioactive glasses, allows its injection and the in situ formation of the composite hydrogel resulting after injection, allowing the composite material to remain at the implantation site.

The presence of particles of bioactive glass(es) in the injectable composition obtained after compositions A and B1, or compositions A and B2, or even compositions A, B1 and C, are mixed, allows immobilization in the bone tissue. The presence of chitosan provides angiogenic properties, stimulates tissue reconstruction and allows larger bone defects to be filled than with the use of bioactive glasses alone. The combination of the two compositions A and B1, or of compositions A and B2, or even of compositions A, B1 and C, makes it possible to exploit both the rheological and biological properties of the chitosan and the bioactive properties of the particles of the selected bioglass(es).

By choosing certain parameters, in particular the pH of composition A, the amount of bioactive glass, the sizes of the bioglass particles, it is possible to control the time between the moment of mixing and the moment of gelation.

The invention also relates to the injectable, glycerophosphate-free compositions, containing particles of one or more bioactive glass(es) suspended in a chitosan aqueous solution, obtained by mixing compositions A and B1, or compositions A and B2, or even compositions A, B1 and C, of the product defined in the present description. Such compositions are described as injectables because they are in a state, prior to the gelation step, which is intended to occur in situ, in particular after injection of the composition into the body of the subject to be treated. In particular, such injectable compositions have a viscosity at a shear rate of 10⁴ s⁻¹ in the range of 0.005 to 200 Pa·s. This viscosity may be measured by capillary rheometry, notably at 22° C., and in particular according to the method described in the publication “Chitosan solutions as injectable systems for dermal filler applications: rheological characterization and biological evidence” by C. Halimi, A. Monternbault, A. Guerry, T. Delair, E. Viguier, R. Fulchiron, L. David. Annual International Conference of the IEEE Engineering in Medicine and Biology Society. IEEE Engineering in Medicine and Biology Society. 2015; 2596-9. The injectable compositions according to the invention advantageously have a pH in the range of 3.1 to 7.4, preferably in the range of 5 to 7. The viscosity and pH characteristics given for the injectable composition preferably correspond to those obtained immediately after mixing the different components used for their preparation, and in particular, compositions A and B1, or compositions A and B2, or even compositions A, B1 and C, as a function of the selected mixing method, and notably within 5 seconds after said mixing.

Advantageously, the injectable compositions are obtained exclusively from compositions A and B1, or from compositions A and B2, or from compositions A, B1 and C. In particular, it is not necessary to add a base, such as soda, to obtain gelation. Gelation of the chitosan present in composition A is obtained by virtue of a bioactive glass present in composition B1. Such an injectable composition is therefore preferably obtained by mixing only compositions A and B1, or only compositions A and B2, or only compositions A, B1 and C. Composition B1 corresponds to particles of one bioactive glass or to a mixture of particles of several bioactive glasses and is therefore in powder form. It is possible, prior to mixing with composition A, to suspend this powder in an aqueous solution, and in particular in water, corresponding to composition C. In this case, this amounts to forming intermediately an aqueous suspension B2 of particles of at least one bioactive glass, the pH of this suspension preferably being in the range of 5.5 to 8, preferably in the range of 6.8 to 7.2.

The invention also relates to the products intended for the preparation of an injectable composition and said injectable compositions, described in the context of the invention, for use in the regeneration of bone tissue, after injection of the injectable composition into a subject, notably in the field of orthopaedics or orthodontics. The subject will be a human being or an animal. In particular, in such a use after injection into a subject, a chitosan hydrogel is formed in vivo. Gelation of the chitosan initially present in composition A is obtained by virtue of a bioactive glass initially present in composition B1.

The invention also concerns a method of regenerating bone tissue in a subject comprising:
the mixing of compositions A and B1, or of compositions A and B2, or of compositions A, B1 and C, leading to the formation of an injectable, glycerophosphate-free composition containing particles of one or more bioactive glass(es) suspended in a chitosan aqueous solution, in particular according to the preparation process described below,
the injection of the resulting injectable composition into said subject in the area where bone tissue regeneration is desired,
the in situ gelation of the injected composition.

The invention also concerns a method of regenerating bone tissue in a subject comprising:
the mixing of a chitosan aqueous solution with a pH in the range of 3 to 7, called composition A, and a powder of particles of one or more bioactive glass(es), called composition B1, in the absence of glycerophosphate, leading to the formation of an injectable, glycerophosphate-free composition containing particles of one or more bioactive glass(es) suspended in a chitosan aqueous solution, in particular according to the preparation process described below,
the injection of the resulting injectable composition into said subject in the area where bone tissue regeneration is desired,
the in situ gelation of the injected composition.

The invention also concerns a method of regenerating bone tissue in a subject comprising:
i) the preparation of an injectable, glycerophosphate-free composition containing particles of one or more bioactive glass(es) suspended in a chitosan aqueous solution, characterized in that it comprises, or even consists exdclusively of, the following successive steps:
have a chitosan aqueous solution with a pH in the range of 3 to 7, called composition A,
a powder of particles of one or more bioactive glass(es), called composition B1,
place composition B1 in aqueous suspension, and preferentially in suspension in water,
mix composition A and the resulting aqueous suspension of particles of at least one bioactive glass, immediately after suspending composition B1, preferably within 20 seconds, and preferentially within 10 seconds, the various compositions and solutions used being glycerophosphate-free,
ii) the injection of the resulting injectable composition into said subject in the area where bone tissue regeneration is desired,
iii) the in situ gelation of the injected composition.

The invention also concerns a packaging device comprising two compositions A and B1 forming a product according to the invention, or even three compositions A, B1 and C. Said device may consist of a single packaging assembly comprising compositions A and B1 packaged separately from each other in two compartments of said packaging assembly, and in particular in an injection system such as a two-compartment syringe.

According to a particular embodiment, the packaging device according to the invention will also comprise composition C, said device which may then be composed of a single packaging assembly comprising at least three independent compartments containing each of the three compositions A, B1 and C, respectively. The device will preferably comprise means to allow the two compositions A and B1, or even the three compositions A, B1 and C, to come into contact and be mixed. In particular, the means allowing the two compositions, or even the three compositions, to come into contact are such that the mixing of said compositions is carried out within said packaging assembly just before injection of the resulting injectable composition. These means may allow sequential mixing, when the device contains compositions A, B1 and C: compositions B1 and C come into contact and mix to form an intermediate composition B2, then compositions B2 and A come into contact and mix. One such device is, for example, a syringe with two or even three compartments.

It is also possible that in the packaging device, compositions A and B1 are packaged separately from each other in two separate packaging units. In the case where a composition C is present, composition C may also be packaged in a separate packaging unit.

The following detailed description of compositions A, B1 and C provides a better understanding of the invention.

Composition A

Composition A contains chitosan. Chitosan is a partially, or even totally deacetylated derivative of chitin. Its different forms are notably characterized by their degree of acetylation (DA) and by their weight-average molecular weight M_{w}.

Preferably, the chitosan of composition A has an average molecular weight M_{w} in the range of 80 to 1000 kg/mol, preferably in the range of 100 to 700 kg/mol and/or the chitosan has a degree of acetylation in the range of 0 to 60%, preferably in the range of 0 to 20%. A degree of acetylation less than or equal to 20% is preferred, to avoid the inflammatory nature of chitosan. It is therefore preferable to use a chitosan with an average molecular weight M_{w} in the range of 100 to 700 kg/mol and a degree of acetylation in the range of 0 to 20%.

In the context of the invention, the weight-average molecular weights M_{w} of chitosan are determined by steric exclusion chromatography, the experimental conditions of which are described in the publication “Physico-chemical studies of the gelation of chitosan in a hydroalcoholic medium” A. MONTEMBAULT, C. VITON, A. DOMARD Biomaterials, 26(8), 933-943, 2005.

The degree of acetylation (DA) is determined using the proton NMR technique, following the methodology of Hirai (Asako Hirai, Hisashi Odani, Akio Nakajima, Polymer Bulletin (1991) Volume: 26, Issue: 1, Publisher: Springer, Pages: 87-94).

Advantageously, the pH of composition A is in the range of 4 to 6.5. Such a pH allows the injected suspension, obtained after mixing of composition A and of that comprising the particles of bioactive glass(es) and stabilization, not to have a pH lower than 6.5, or even preferably lower than 6.8, the pKa of the amine of the glucosamine residues of the chitosan being 6.2. A lower pH of solution A dose to 4 can nevertheless be envisaged, if the time between the mixing of composition A and of that comprising the particles of bioactive glass(es) and the injection of the injectable composition then obtained is sufficient to allow the pH to rise above 6.5.

In particular, composition A comprises an amount of acid necessary to protonate the amine functions present on the chitosan, said acid being preferably acetic or hydrochloric acid.

Notably, composition A will comprise an amount of acid just necessary to protonate the amine functions of the glucosamine residues of the chitosan. The acid will therefore preferably be present in an amount equimolar to the glucosamine residues of the chitosan.

In composition A, the chitosan will therefore be in a protonated form, with respect to its amine functions, which will therefore be in the form —NH₃⁺. These —NH₃⁺ groups create repulsions on the chitosan chains, which will then be in deployed form. The polymer is therefore dissolved in aqueous solution.

The pH of the chitosan aqueous solution may be adjusted by incorporating acetic acid into the chitosan aqueous solution or by incorporating soda. Another acid, such as hydrochloric acid, could also be used.

Preferably, composition A comprises a chitosan concentration of 0.5 to 6% (w/w), preferably of 1 to 4% (w/w). This concentration is expressed in % w/w, i.e. the % that the weight of chitosan represents relative to the weight of composition A. With the lowest concentrations of chitosan, the solution is less viscous, which allows better control of the homogeneity of the final gel after mixing with the particles of bioactive glass(es). The gel obtained in situ with the lowest concentrations of chitosan is more easily colonizable by cells.

Particularly preferably, the pH of composition A is in the range of 4 to 6.5 and contains a chitosan concentration of 1 to 4% (w/w).

Compositions B1 and B2

Advantageously, composition B1 comprises particles of a bioactive glass that contains at least SiO₂, Na₂O, CaO and P₂O₅.

Many other types of glasses can be used. The glass(es) will be selected so as to obtain, after mixing of the compositions of chitosan and of selected bioactive glass(es), an increase in pH for the mixture, within a given time. In particular, it is sought to obtain for the injectable composition, at a time t which is in the range of 5 to 20 minutes, a pH in the range of 6.8 to 7.4.

In particular, said bioactive glass contains 30 to 70 wt % SiO₂, 10 to 50 wt % CaO, 5 to 40 wt % Na₂O and 2 to 20 wt % P₂O₅.

Notably, a bioactive glass with the following composition may be used: 40 to 60 wt % SiO₂, 10 to 30 wt % CaO, 10 to 35 wt % Na₂O and 2 to 8 wt % P₂O₅. Such a bioactive glass is 45S5 glass (marketed notably by Biometric or Mo-Sci Corporation, or in the form of medical devices by the companies Noraker, Novabone, etc.). This bioactive glass is only moderately used by practitioners, mainly because its shaping remains very limited. The chitosan-based solution described in the present invention will, after gelation, provide a matrix support for the bioactive glass particles, which addresses previous problems.

Many other glass compositions may be used: what is important is that bioactive glass contains a sufficient amount of oxide to release cations that will be able to exchange with the protons in the environment. Such an exchange must be possible when the bioactive glass is in aqueous suspension, and in particular when it is mixed with the chitosan solution. Such cations may be alkali or alkaline-earth metal cations, for example Ca²⁺, Na⁺, K⁺, Mg²⁺ Ba²⁺ or Sr²⁺ ions. The composition of said bioactive glass will therefore contain at least one of the following components: CaO, Na₂O, K₂O, MgO, or SrO. The presence of strontium, which is a radiopaque component, will allow the composition to be localized in vivo, after injection and gelation. The glass will also generally contain SiO₂. This SiO₂ part will preferably be selected so as not to prevent the mobility of cations within the bioactive glass, which are intended to exchange with the protons of the chitosan and thus allow their release. In particular, composition B1 will comprise particles of a bioactive glass containing 40 to 60 wt % SiO₂ and 2 to 8 wt % P₂O₅ and at least one component, or even 2, 3 or 4, selected from Ca²⁺, Na⁺, K⁺, Mg²⁺, Sr²⁺, B³⁺, with all these components representing 30 to 55 wt %. Conventionally, the composition of a bioactive glass is given in this text in wt %, based on the total weight of said glass, and the sum of the % of each of the given components will be either 100% or at least 95%, which does not necessarily exclude the presence of other components.

The bioactive glass causes an increase in pH when mixed with the chitosan aqueous solution (composition A). This increase in pH means that the —NH₃ functions initially present on the chitosan chains will no longer be in protonated form, but in —NH₂ form, thus allowing the formation of hydrogen bonds and hydrophobic bonds between the polymer chains, or even within the same chain, ultimately leading to gelation.

It is also possible to use a combination of several bioactive glasses: for example, a first bioactive glass mainly used for obtaining gelling capacity and a second bioactive glass mainly used for its osteoinductivity, or which will lead to the release of ions, with delayed effect. The presence of SrO to promote localization of the implant may be provided for in one or the other of the bioactive glass compositions. Preferably, the first bioactive glass may be one of those described above, notably a bioactive glass containing 40 to 60 wt % SiO₂, 10 to 30 wt % CaO, 10 to 35 wt % Na₂O and 2 to 8 wt % P₂O₅. The second bioactive glass may contain 60 to 75 wt % P₂O₅, 6 to 35 wt % CaO, 5 to 12 wt % Na₂O, 0 to 18 wt % K₂O and 0 to 8 wt % Al₂O₃.

Advantageously, the bioactive glass particles have a size in the range of 3 to 500 μm, and notably in the range of 60 to 250 μm.

Of course, the composition of the selected bioactive glass or mixture of bioactive glass(es) and the particle size of said bioactive glass has an influence on the bioactivity of the product and on the gelation of the composition. The bioactive glass particles may be obtained by grinding bioactive glass ingot and the desired size will be obtained by passing the resulting particles through sieves, using techniques well known to the skilled person. The size mentioned in the context of the invention, unless otherwise specified, therefore corresponds to the largest dimension of a particle. Choosing a finer grain size results in faster gelation. Also, if the composition of a bioactive glass contains fewer releasable cations, the skilled person will be able, by reducing the particle size of said bioactive glass, to increase the quantity of released cations and thus obtain the desired effect with respect to chitosan.

Composition B1 in the form of a powder of particles of one or more bioactive glass(es) may be mixed directly with composition A, to form the injectable formulation. It is also possible to form intermediately an aqueous suspension of particles of one or more bioactive glass(es) (composition B2). When a composition B2 in the form of an aqueous suspension of particles of one or more bioactive glass(es) is formed intermediately, it will preferably be formed shortly before compositions A and B2 are mixed. In particular, the suspension corresponding to composition B2 will be formed less than 20 seconds before, preferably less than 10 seconds before, the latter is mixed with composition A. Indeed, the pH of an aqueous suspension of bioglasses changes over time. In the context of the invention, the pH of a suspension B2 increases over time, due to cation exchange with the water. Also, the pH of composition B2 given in the context of the invention, which is in the range of 5.5 to 8, preferentially in the range of 6.8 to 7.2, preferably corresponds to the pH of the suspension measured immediately after its formation, notably within 5 seconds after its constitution.

Advantageously, the composition B2 comprises at least 15% (w/w), preferably 30 to 60%, particles of one or more bioactive glass(es). These %, expressed in % w/w, correspond to the % that the weight of bioactive glass(es) represents relative to the weight of composition B2.

When a composition B2 corresponding to an aqueous suspension of one or more bioactive glasses is formed intermediately, it will be obtained by introducing bioactive glass particles or a mixture of bioactive glass particles of composition B1 into composition C. Preferably, composition C is water, but it is not excluded that it also comprises calcium phosphates, provided that it meets the pH conditions which must be in the range of 5.5 to 8, preferably in the range of 6.8 to 7.2.

### Other Characteristics of the Product According to the Invention and Packaging Device

The aqueous solutions or suspensions used in the present invention for compositions A, C and B2 will be physiologically acceptable. Preferably, such solutions or suspensions will be prepared in water, and notably in deionized or ultrapure water.

The proportions of composition A and composition B1, or even composition C when present, will be adjusted by the skilled person, notably to have the desired viscosity. Advantageously, the weight of composition B1 or of compositions B1 and C, relative to the total weight of compositions A and B1 or of compositions A, B1 and C, respectively, will be in the range of 20% to 90%, preferably in the range of 40% to 80%. The amount of compositions B1 and C may be adjusted, by the skilled person, according to the weight proportion that the bioglass particles represent in composition B2 obtained after mixing, in order to obtain the desired injectability for the final composition. The amount of bioactive glass will be adjusted to control the time required to obtain a gel. Increasing the amount of bioactive glass allows a higher pH to be obtained more quickly, and therefore faster gelation.

All the parameters listed above will be adjusted by the skilled person so as to make the time required for gelation compatible with the time required for injection. Preferably, gelation will occur between 5 and 20 minutes after compositions A and B1, or compositions A and B2, or compositions A, B1 and C, are mixed, as a function of the product according to the invention and of the selected mixing method.

According to a particular embodiment of the product according to the invention, compositions A and B1 are packaged separately from each other in two compartments of a single packaging assembly, and in particular in an injection system such as a two-compartment syringe. When the product according to the invention also comprises a composition C, compositions A, B1 and C are advantageously packaged separately from each other in three compartments of a single packaging assembly, and in particular in an injection system such as a three-compartment syringe.

According to another particular embodiment of the product according to the invention, compositions A and B1 are packaged separately from each other in two separate packaging units. When the product according to the invention also comprises a composition C, compositions A, B1 and C are advantageously packaged separately from each other in three separate packaging units.

All combinations are possible, the first being nevertheless preferred:
composition C and B1 in a single packaging assembly comprising compositions C and B1 packaged separately from each other in two compartments of said packaging assembly, and composition A packaged separately in another packaging unit;
composition A and B1 in a single packaging assembly comprising compositions A and B1 packaged separately from each other in two compartments of said packaging assembly, and composition C packaged separately in another packaging unit;
composition A and C in a single packaging assembly comprising compositions A and C packaged separately from each other in two compartments of said packaging assembly, and composition B1 packaged separately in another packaging unit.

In these different cases, composition B1 is in a dry form, i.e. in the form of a powder of particles of one or more bioactive glasses. With such a composition B1, and in particular a composition B1 consisting exclusively of particles of one or more bioactive glasses, the product according to the invention is particularly stable.

For the rest, the preferred characteristics of compositions A and B1 listed above apply, also in a preferred manner, to the packaging device according to the invention.

In particular, the packaging device according to the invention will combine:
a composition A having a pH in the range of 4 to 6.5 and containing a chitosan concentration of 1 to 4% (w/w),
a composition B1 corresponding to particles of one or more bioactive glass(es), notably in powder form.

The amounts of composition A and B1 will be selected so that the weight of composition B1, relative to the total weight of the resulting injectable composition, is in the range of 20% to 90%, preferably in the range of 40% to 80%.

Of course, the chitosan and the bioglasses will advantageously correspond to those presented as preferred in the previous sections relating to compositions A and B1. When the packaging device comprises a composition C, it will preferably be water.

Process for Preparing the Injectable Composition

The invention relates to a process for preparing an injectable, glycerophosphate-free composition containing particles of one or more bioactive glass(es) suspended in a chitosan aqueous solution, said process comprising, or even consisting exclusively in, the mixing of:
a chitosan aqueous solution with a pH in the range of 3 to 7, called composition A, and
a powder of particles of one or more bioactive glass(es), called composition B1,

in the absence of glycerophosphate.

The invention also relates to a process for preparing an injectable, glycerophosphate-free composition containing particles of one or more bioactive glass(es) suspended in a chitosan aqueous solution, characterized in that it comprises, or even consists exclusively of, the following successive steps:
have a chitosan aqueous solution with a pH in the range of 3 to 7, called composition A,
a powder of particles of one or more bioactive glass(es), called composition B1,
place composition B1 in aqueous suspension, and preferentially in suspension in water,
mix composition A and the resulting aqueous suspension of particles of at least one bioactive glass, immediately after suspending composition B1, preferably within 20 seconds, and preferentially within 10 seconds,
   the various compositions and solutions used being glycerophosphate-free.

Preferably, the injectable composition is prepared using compositions A and B1, compositions A and B2, or even compositions A, B1 and C, contained in a product according to the invention as previously defined.

Preferably, the injectable composition according to the invention does not contain genipin, glutaraldehyde or formaldehyde. More generally, the injectable composition according to the invention does not contain a cross-linking agent. Also, such products will not be present in the various compositions and solutions used in the processes according to the invention.

To obtain the injectable composition, compositions A and B1, compositions A and B2, or even compositions A, B1 and C, may be mixed by any appropriate addition or injection method, in particular.

Composition A may be mixed directly with a composition B1 consisting of a powder of particles of bioactive glass(es), for example, by putting the bioglass particles in powder form directly into the chitosan solution. It is also possible to put the bioglass particles in aqueous suspension before mixing with the chitosan solution (composition A). This suspension may be carried out with a composition C, present in the product according to the invention, or with an aqueous solution, and in particular water, external to the product according to the invention. In both cases, this suspension will be carried out just before the mixing with composition A, for example, less than 20 seconds before, and preferentially less than 10 seconds before. In this case, the product according to the invention may comprise three compartments, disposed in a single packaging unit or in several packaging units: one containing composition A, another containing composition B1 of bioactive glass(es), and the third containing water (composition C).

According to a first variant embodiment, the process for preparing an injectable composition according to the invention comprises, or even consists exclusively of, the following successive steps:
have a composition A, as defined in the context of the invention,
have a composition B1,
mix compositions A and B1.

In particular, according to a second variant embodiment, the process for preparing an injectable composition according to the invention comprises, or even consists exclusively of, the following successive steps:
have a composition A, as defined in the context of the invention,
have a composition B1,
place composition B1 in aqueous suspension, and preferentially in suspension in water,
mix composition A and the resulting suspension, immediately after suspending composition B1, preferably within 20 seconds, and preferentially within 10 seconds.

The glass suspension formed will preferably comprise at least 15% (w/w), preferably 30 to 60%, particles of one or more bioactive glass(es). Advantageously, the suspension of composition B1 corresponds to the formation of a composition B2, having a pH in the range of 5.5 to 8, preferentially in the range of 6.8 to 7.2, when said pH Is measured immediately after its formation, notably within 5 seconds after its constitution.

Preferably, the suspension formed is obtained by suspending composition A in composition C as defined in the context of the invention.

In such processes, the mixture may be made more homogeneous by means of mechanical agitation or dynamic mixing (dynamic flow of one composition into another, notably of composition A into composition B1, or vice versa, or of suspension B2 into composition A in particular).

Here again, the preferred characteristics of compositions A and B1 listed above apply, also in a preferred manner, to the preparation process according to the invention.

In particular, the following will be used in the process according to the invention:
a composition A having a pH in the range of 4 to 6.5 and containing a chitosan concentration of 1 to 4% (w/w),
a composition B1 corresponding to particles of one or more bioactive glass(es), notably in powder form.

The amounts of composition A and B1, or even the amounts of aqueous solution and in particular water, to prepare an intermediate suspension, will be selected so that the weight of composition B1, relative to the total weight of the resulting injectable composition, is in the range of 20% to 90%, preferably in the range of 40% to 80%.

Of course, here again, the chitosan and the bioglasses will advantageously correspond to those presented as preferred in the previous sections relating to compositions A and B1.

The invention also relates to the injectable compositions obtainable by the preparation processes described in the context of the invention.

The resulting composition is intended to be injected into a subject. Preferably, the mixing of compositions A and B1, or even of compositions B1 and C forming a composition B2, then of compositions A and B2, or of compositions A, B1 and C, will take place just before injection of the resulting injectable composition into a subject, notably less than 1 minute before, preferably less than 30 seconds before. Of course, the injection must take place before gelation.

The following examples, with reference to the appended Figures, are intended to illustrate the invention but are in no way limiting.

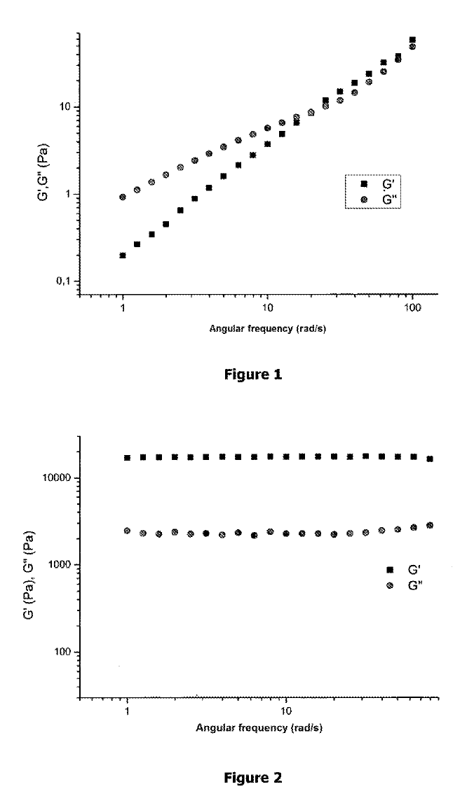

FIG. 1 shows changes in G′ and G″ moduli as a function of frequency for a chitosan solution with a concentration of 0.375% w/v (imposed deformation: 0.5%).

FIG. 2 shows changes in G′ and G″ as a function of angular frequency, 2 minutes 30 seconds after mixing (imposed deformation: 0.5%).

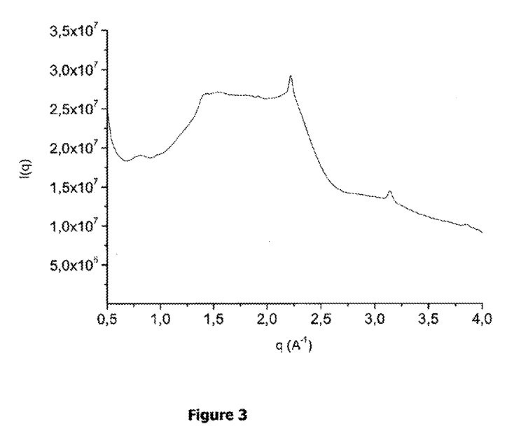

FIG. 3 shows the X-ray diffractogram obtained after immersion of a hydrogel formed from a suspension containing bioglasses and chitosan for 4 days in SBF at 37° C.

### METHOD FOR DETERMINING PH

The pH value of the mixture of compositions is advantageously measured using a contact pH meter (Mettler-Toledo AG, Switzerland).

Method for Determining Viscosity

The viscosity measurement is performed at high shear rate (typically 10⁴ s⁻¹) and is obtained by capillary rheometry according to the method described in the publication “Chitosan solutions as injectable systems for dermal filler applications: rheological characterization and biological evidence” by C. Halimi, A. Monternbault, A. Guerry, T. Delair, E. Viguier, R. Fulchiron, L. David. Annual International Conference of the IEEE Engineering in Medicine and Biology Society. IEEE Engineering in Medicine and Biology Society. 2015; 2596-9.

Method for Adjusting the Granulometry (Particle Size) of the Bioglasses

The resulting glasses are ground using an agate mortar/pestle or a tungsten carbide ball mill.

The resulting powders are then dry-sieved through a column of vibrating sieves of variable mesh diameters (63 μm, 90 μm, 125 μm, 150 μm, 200 μm, 250 μm, 315 μm and/or 500 μm).

In order to remove the agglomerated fine particles not corresponding to the desired particle sizes, the various batches obtained after separation by sieving are each immersed in a beaker containing 200 ml of acetone and placed in an ultrasonic tank. Large particles fall to the bottom of the beaker, while fines are removed with acetone. This procedure is repeated until the acetone is dear (free of fine particles). The batches of powder of defined particle size are then dried in an oven at 92° C. for 20 to 30 minutes.

Method for Obtaining the Diffractogram

The measurement of diffractograms is performed using a laboratory X-ray diffractometer, using a Cu Kα X-ray source and diffraction angles comprised between 3 and 60 degrees 2θ (i.e. moduli of the scattering vector q between 0.2 and 4), and in Bragg-Brentano geometry.

1. Demonstration of Gel Formation

A 0.75 wt % chitosan aqueous solution is prepared. To that end, chitosan is dispersed in a volume of deionized water. Acetic acid is added so as to allow stoichiometric protonation of the amine functions of the chitosan. The chitosan used in this example has the following structural characteristics: degree of acetylation of 5%, molecular weight M_{w} of 550 000 g/mol and a polydispersity index Iₚ of 1.4.

At the same time, a bioactive glass powder is dispersed in deionized water, the volume of which corresponds to the volume of the chitosan solution. More precisely, it is 45S5 glass with the following weight composition: 45% SiO₂, 24.5% CaO, 24.5% Na₂O and 6% P₂O₅. The weight of glass incorporated is equal to half the weight corresponding to the chitosan solution.

Thus, the amounts used in this example are as follows:
750 mg of 0.75% (w/w) chitosan solution,
375 mg of 45S5 glass particles dispersed in 750 mg of deionized water.

The glasses used and presented in the context of this invention were prepared at the Laboratoire Charles Coulomb of the Universite de Montpellier. The bioglasses were elaborated from a mixture of the following raw materials: Al(OH)₃, H₂NaPO₄, CaCO₃, H₂(NH)₄PO₄, SiO₂ and K₂CO₃ in appropriate proportions as a function of the desired composition. In the case of elaboration of 45S5 glass, the raw materials used are Na₂CO₃ (Prolabo), CaCO₃ (MerckKGaA), H₂(NH)₄PO₄ (Prolabo), and SiO₂ (Prolabo). Each mixture was melted in a platinum crucible. The resulting melt was cast in a steel mould and the glass obtained after air cooling was annealed 1 h at a temperature slightly above its glass transition temperature and then cooled very slowly to room temperature.

Each glass ingot formed was then ground and sieved to obtain powders of different particle sizes. The particle size of the glass powder used in the context of this example is comprised between 200 and 315 microns and is selected by means of the sieves used.

The formulation combining the dispersion of bioglass and the chitosan solution was stirred to obtain a homogeneous system. In the end, the chitosan concentration in the mixture is 0.375% (w/v).

When the mixture is prepared under stirring, a spontaneous gelation mechanism occurs leading to the formation of a physical hydrogel containing a glass mineral part.

Fifteen minutes after mixing, the pH of the composite material stabilizes at 7.4.

2. Rheological Characteristics

This section presents the rheological characterizations that were carried out in order to demonstrate the gelling capacity of the bioglasses when mixed with a chitosan solution.

A 0.75 wt % chitosan aqueous solution was prepared, as before.

At the same time, bioactive glasses in powder form are dispersed in deionized water whose volume is equal to the volume of the chitosan solution. More precisely, it is a mixture of glasses composed of ⅔ 45S5 and ⅓ Na₁₆Ca₃₀P₅₀Al₄ glass (in weight proportions). Na₁₆Ca₃₀P₅₀Al₄ glass (referred to here as “phosphate glass”) is composed of 9.8% Na₂O, 16.5% CaO, 69.7% P₂O₅ and 4% Al₂O₃ (% w/w). This glass was elaborated by following the same procedure as that described above in the paragraph 1. Demonstration of gel formation, i.e. from a mixture of the following raw materials: AKOH)₃ (FlukaBioChemika), H₂NaPO₄ (FlukaChemie) CaCO₃ (MerckKGaA), H₂(NH)₄PO₄ (Prolabo) in appropriate proportions, melted in a platinum crucible. The glass obtained by casting the melt followed by annealing and slow cooling was then ground and sieved, as was described for composition A in the paragraph Method for adjusting the granulometry (particle size) of the bioglasses. The particle size of the 45S5 and Na₁₆Ca₃₀P₅₀Al₄ glass powders used in the context of this example is between 90 and 150 microns.

The weight of the glasses incorporated is equal to half the weight of the chitosan solution.

Thus, the amounts used in this example are as follows:
750 mg of 0.75% (w/w) chitosan solution,
375 mg of glasses dispersed in 750 mg of deionized water.

This 375 mg is made up of 250 mg of 45S5 glass and 125 mg of phosphate glass.

The particle size of the glasses used in the context of this example is comprised between 90 and 150 microns and is selected by means of the sieves used.

The formulation combining the dispersion of bioglasses and the chitosan solution is stirred so as to obtain a homogeneous system. In the end, the chitosan concentration in the mixture is 0.375% (w/v).

In order to facilitate homogenization of the formulation, a system of two connected syringes is used. A connector connects the two syringes, one containing the chitosan solution, the other the suspension of bioglasses. Mixing is thus achieved by sending the contents back and forth between the two syringes several times.

During the homogeneous mixing process, a spontaneous gelation mechanism occurs leading to the formation of a physical hydrogel containing a glass mineral part.

The corresponding sol-gel transition was studied by rheometry.

These tests were performed on an AR 2000 rheometer (TA Instruments), using a plate-plate geometry with a 25 mm diameter plate and a sample containment system to maintain a water-saturated atmosphere and thus limit evaporation of the water present in the sample. All tests were performed at room temperature (25° C.).

A rheological characterization was first performed on a 0.375% (w/v) concentrated chitosan solution not containing glass. The curves obtained for a glass-free formulation are presented in FIG. 1 and show rheological behaviour corresponding to the solution state. The shape of the curves is characteristic of viscoelastic solutions with G′<G″ at low frequencies and G′>G″ at high frequencies, G′ and G″ being defined respectively as the elastic and viscous moduli of the sample.

A rheological characterization was then performed on the same solution, but into which a suspension of bioglass particles dispersed in deionized water was incorporated (suspension of 45S5 glass/phosphate glass and introduced in the proportion described above).

FIG. 2 shows the rheological curves obtained 2 minutes 30 seconds after mixing. These curves show rheological behaviour corresponding to a gel state, with G′ and G″ moduli values such that G′>10 G″, regardless of the angular frequency.

This gelation property is fully compatible with the development of injectable systems.

The presence of bioglasses within the solution causes an increase in pH in the medium and induces deprotonation of the amine sites of the chitosan, which allows gelation of the polymer solution.

3. Study of Changes in the Resulting Gels Upon Contact with a Solution Having a Composition Close to that of Human Physiological Fluids (“Simulated Body Fluid” Solution)

A study was carried out to verify that when the bioglass particles are present in the hydrogel, the bioglasses retain their bioactivity properties.

In order to evaluate this bioactivity, the elaborated gels were immersed in a “Simulated Body Fluid” (SBF) solution. This SBF solution has an ion concentration dose to that of human physiological fluids maintained under physiological temperature and pH conditions (37° C. and 7.5) [T. Kokubo, H. K., S. Sakka, T. Kitsugi and T. Yamamuro. Solutions able to reproduce in vivo surface-structure changes in bioactive glass-ceramic A-W. J. Biomed. Mater. Res. 1990, vol. 24, 721-734].

The composites studied here had the following composition:
750 mg of 0.75% (w/w) chitosan solution in deionized water,
375 mg of glasses dispersed in 750 mg of deionized water, this 375 mg comprising 250 mg of 45S5 glass and 125 mg of phosphate glass described in section 2 above.

The particle size of the glasses used in the context of this example was comprised between 90 and 150 microns.

Thus, after immersing the composite materials in an SBF solution for two days, the water from the resulting sample was removed by freeze-drying and analysed by X-ray diffraction. The X-ray diffractogram presented in FIG. 3 shows the formation of a crystalline phase, calcite. The arrows point to the diffraction peaks characteristic of calcite. In the case of the bioglasses alone, it is generally observed that the calcite that precipitates initially is then transformed into hydroxyapatite: this phase may thus be considered under these conditions as a precursor of carbonated hydroxyapatite [Lefebvre, L., Développement de bioverres poreu pour application à I'ortopddie et à I'ingénienre tissulaire. 2007, INSA Lyon].

In the case of the bioglasses incorporated into the hydrogel, the phenomena of ion exchanges with the environment are therefore comparable to those observed in the case of bioglasses alone. These experiments show that the behaviour of the bioglasses has not been modified when incorporated into a chitosan hydrogel and that these bioglasses have retained their bioactivity properties.

1-38. (canceled)

39. A product intended for the preparation of an injectable composition containing, separately from each other, at least one first composition A and one second composition B1, and optionally a third composition C, wherein:
composition A is a chitosan aqueous solution with a pH in the range of 3 to 7, the chitosan having an average molecular weight M_{w} in the range of 80 to 1000 kg/mol and a degree of acetylation in the range of 0 to 60%,
composition B1 consists in particles of one or more bioactive glass(es),
composition C, when present, is water or an aqueous solution with a pH in the range of 5.5 to 8;
said product not containing glycerophosphate and wherein
the weight of composition B1 relative to the total weight of compositions A and B1, or the weight of compositions B1 and C relative to the total weight of compositions A, B1 and C, is in the range of 20% to 90%.

40. The product according to claim 39, wherein it does not contain genipin, glutaraldehyde or formaldehyde.

41. The product according to claim 39, wherein the pH of composition A is in the range of 4 to 6.5.

42. The product according to claim 39, wherein composition A comprises a chitosan concentration of 0.5 to 6% (w/w).

43. The product according to claim 39, wherein the chitosan has an average molecular weight M_{w} in the range of 100 to 700 kg/mol.

44. The product according to claim 39, wherein the chitosan has a degree of acetylation in the range of 0 to 20%.

45. The product according to claim 39, wherein composition B1 comprises particles of a bioactive glass that contains at least SiO₂, Na₂O, CaO and P₂O₅.

46. The product according to claim 39, wherein said bioactive glass contains 30 to 70 wt % SiO₂, 10 to 50 wt % CaO, 5 to 40 wt % Na₂O and 2 to 20 wt % P₂O₅.

47. The product according to claim 39, wherein composition B1 comprises particles of a first bioactive glass containing 40 to 60 wt % SiO₂, 10 to 30 wt % CaO, 10 to 35 wt % Na₂O and 2 to 8 wt % P₂O₅ and particles of a second bioactive glass containing 60 to 75 wt % P₂O₅, 6 to 35 wt % CaO, 5 to 12 wt % Na₂O, 0 to 18 wt % K₂O and 0 to 8 wt % Al₂O₃.

48. The product according to claim 39, wherein the bioactive glass particles have a size in the range of 3 to 500 μm.

49. The product according to claim 39, wherein it also comprises a composition C wherein composition C is present in an amount such that composition B1 represents at least 15% (w/w) of the total weight of compositions B1 and C.

50. The product according to claim 39, wherein the weight of composition B1 relative to the total weight of compositions A and B1, or of compositions B1 and C relative to the total weight of compositions A, B1 and C is in the range of 40% to 60%.

51. The product according to claim 39, wherein compositions A and B1 are packaged separately from each other in two compartments of a single packaging assembly.

52. The product according to claim 39, wherein compositions A and B1 are packaged separately from each other in two separate packaging units.

53. An injectable composition corresponding to a suspension of one or more bioactive glass(es) in a chitosan aqueous solution, obtained by mixing compositions A and B1, by mixing compositions A, B1 and C, or by mixing compositions B1 and C, to form an intermediate composition B2, then B2 and C, of the product as defined in claim 39.

54. The injectable composition according to claim 53, wherein it has a viscosity at a shear rate of 10⁴ s⁻¹ in the range of 0.005 to 200 Pa·s.

55. A process for preparing an injectable, glycerophosphate-free composition containing particles of one or more bioactive glass(es) suspended in a chitosan aqueous solution, said process comprising the mixing of composition A and composition B1 as defined in claim 39 in the absence of glycerophosphate, wherein the weight of composition B1 relative to the total weight of compositions A and B1 is in the range of 20% to 90%.

56. A process for preparing an injectable, glycerophosphate-free composition containing particles of one or more bioactive glass(es) suspended in a chitosan aqueous solution, comprising the following successive steps of:
providing composition A as defined in claim 39,
providing composition B1 as defined in claim 39,
providing water or an aqueous solution with a pH in the range of 5.5 to 8,
mixing composition B1 with water or with the aqueous solution to form an aqueous suspension of particles of at least one bioactive glass, and
immediately after mixing composition A aqueous suspension,
the various compositions and solutions used being glycerophosphate-free, and wherein the weight of the aqueous suspension to the total weight of composition A and the aqueous suspension is in the range of 20% to 90%.

57. The process according to claim 56, wherein the aqueous suspension of particles of at least one bioactive glass has a pH in the range of 5.5 to 8.
